(19) United States
(12) Patent Application Publication
(10) Pub. No.: US 2023/0176397 A1
(43) Pub. Date: Jun. 8, 2023

(54) BLINK PROMOTION DEVICE AND BLINK PROMOTION METHOD

(71) Applicant: Nippon Telegraph and Telephone Corporation, Tokyo (JP)

(72) Inventors: Arinobu NIIJIMA, Musashino-shi, Tokyo (JP); Toki TAKEDA, Musashino-shi, Tokyo (JP); Takashi SATO, Musashino-shi, Tokyo (JP)

(21) Appl. No.: 18/013,661

(22) PCT Filed: Jul. 3, 2020
(86) PCT No.: PCT/JP2020/026132

§ 371 Date: Dec. 29, 2022

(51) Int. Cl. G02C 5/00; A61B 3/113

(52) U.S. Cl. CPC G02C 5/001; A61B 3/113

(57) ABSTRACT

A blink acceleration device (1) includes: a number of blinks calculation unit (141) that calculates the number of blink operations per unit time of a monitoring target person; and a tactile stimulus operation unit (12) that performs an operation of stimulating eyelashes as a tactile stimulus operation for the monitoring target person when the number calculated by the number of blinks calculation unit is less than a preset threshold.

### TECHNICAL FIELD

The present invention relates to a blink acceleration device and a blink acceleration method.

### BACKGROUND ART

In recent years, with the spread of tele-working, work using devices such as personal computers and smart devices has increased. As the work using these devices increases, the time during which a person who works continues to view a display becomes longer, and health impairment such as computer vision syndrome and visual display terminal syndrome (VDT syndrome) caused by this becomes a problem.

For example, it is reported that the number of blinks of a worker is reduced to about ⅓ to ¼ of the normal number when the worker intensively performs the work while watching a display, and there is a risk of leading to dry eye which is one of symptoms of the above-described syndrome.

In order to prevent dry eye symptoms, it is effective to blink about once every three seconds. In view of this, a system has been proposed in which the number of blinks is counted by analyzing imaging information obtained by imaging the vicinity of the eyes of a worker with a web camera, and when the number of blinks within a certain period of time is equal to or less than a predetermined value, a pop-up display is performed on a display being used to accelerate blinking (Non Patent Literature 1). As a method for accelerating blinking, it is conceivable to emit a warning sound in addition to performing pop-up display on the display. As another method, a method has been proposed in which contact stimulation by a servomotor or wind stimulation by a microblower is presented in the vicinity of the outer corner of the eye to cause corneal reflex, thereby accelerating blinking (Non Patent Literature 2).

### CITATION LIST

### Non Patent Literature

Non Patent Literature 1: Yuto HOJO, et al. “PC User's Blink Promotion System Using Translucence Pop-Up Alert on Cursor” [C] Proceedings of the 26 Institute of Electrical Engineers of Japan Institute of Electronics, Information, and Systems Conference (2014): 656-657.

Non Patent Literature 2: Dementyev, Artem, and Christian Holz. “DualBlink: a wearable device to continuously detect, track, and actuate blinking for alleviating dry eyes and computer vision syndrome.” Proceedings of the ACM on Interactive, Mobile, Wearable and Ubiquitous Technologies 1.1 (2017):1-19.

### SUMMARY OF INVENTION

### Technical Problem

However, when a worker is working intensively, if a pop-up display appears on a display, the visual sense of the worker is obstructed, and if a warning sound is emitted, the hearing is obstructed, which hinders the work. Furthermore, since the worker has to consciously blink in response to the stimulus, there is a problem that this also leads to an increase in the cognitive load of the worker. Furthermore, since sufficient stimulation intensity is necessary to cause corneal reflex by contact stimulation or wind stimulation, there is a problem that the voltage for driving the actuator increases. For example, in the wind stimulation of Non Patent Literature 2, a voltage of 24 V is applied.

The present invention has been made in view of the above circumstances, and an object thereof is to provide a blink acceleration device and a blink acceleration method capable of accelerating blinking with low power consumption without applying a load to a user as much as possible.

### Solution to Problem

In order to achieve the above object, one aspect of the present invention is a blink acceleration device including: a number of blinks calculation unit that calculates the number of blink operations per unit time of a monitoring target person; and a tactile stimulus operation unit that performs an operation of stimulating eyelashes as a tactile stimulus operation for the monitoring target person when the number calculated by the number of blinks calculation unit is less than a preset threshold.

An aspect of the present invention is a blink acceleration method, in which a blink acceleration device calculates the number of blink operations per unit time of a monitoring target person, and when the calculated number is less than a preset threshold, performs an operation of stimulating eyelashes as a tactile stimulus operation for the monitoring target person.

Reflection (eyelash reflex) to a tactile stimulus to eyelashes can be induced with a lower stimulation intensity than that in a case of corneal reflex by a tactile stimulus to the vicinity of the outer corners of eyes, so that voltage applied to a tactile actuator for presenting a tactile stimulus can be suppressed.

### Advantageous Effects of Invention

According to the present invention, it is possible to provide a blink acceleration device and a blink acceleration method capable of accelerating blinking with low power consumption without applying a load to a user as much as possible.

### BRIEF DESCRIPTION OF DRAWINGS

FIG. 1 is a block diagram illustrating a configuration of a blink acceleration device according to an embodiment of the present invention.

FIG. 2 is an external view illustrating glasses on which the blink acceleration device of the embodiment is mounted.

FIG. 3 is a flowchart illustrating an operation of the blink acceleration device according to the embodiment.

FIG. 4 is a graph illustrating a temporal change in a measurement value of a distance to human skin by a photo reflector as an operation state acquisition unit according to the embodiment.

FIG. 5 is a hardware configuration example.

### DESCRIPTION OF EMBODIMENTS

### Configuration of Blink Acceleration Device According to One Embodiment

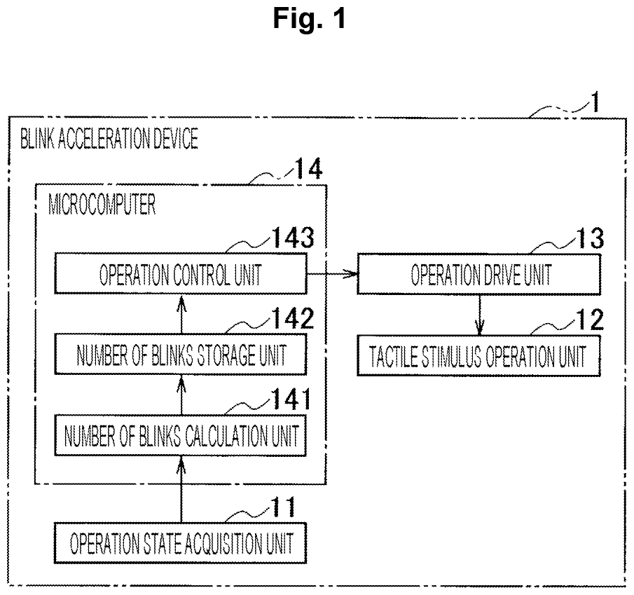

A configuration of a blink acceleration device according to an embodiment of the present invention will be described with reference to FIG. 1. A blink acceleration device 1 according to the present embodiment includes an operation state acquisition unit 11, a tactile stimulus operation unit 12, an operation drive unit 13, and a microcomputer 14.

The operation state acquisition unit 11 acquires information indicating the operating state of the skin near the eyes of the user who is a monitoring target person. The tactile stimulus operation unit 12 performs an operation of stimulating eyelashes as a tactile stimulus operation for the user. The operation drive unit 13 drives the operation of the tactile stimulus operation unit 12.

The microcomputer 14 includes a number of blinks calculation unit 141, a number of blinks storage unit 142, and an operation control unit 143. The number of blinks calculation unit 141 calculates the number of blink operations of the user per unit time on the basis of the information acquired by the operation state acquisition unit 11. The number of blinks storage unit 142 stores information on the number of blink operations calculated by the number of blinks calculation unit 141.

When the number stored in the number of blinks storage unit 142 is less than a preset threshold, the operation control unit 143 determines a parameter related to the operation of the operation drive unit 13 and causes the corresponding operation to be performed.

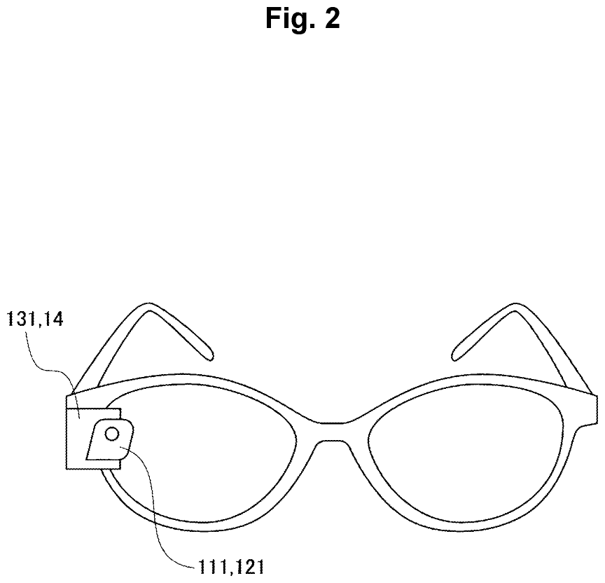

In the present embodiment, a case where the blink acceleration device 1 is mounted beside the lens of glasses 2 as illustrated in FIG. 2, a photo reflector 111 is used as the operation state acquisition unit 11, a microblower 121 is used as the tactile stimulus operation unit 12, and a motor driver 131 is used as the operation drive unit 13 will be described as an example. The photo reflector 111 is a sensor that determines a distance to an object by irradiating the object with light and detecting reflected light. The microblower 121 is a device that presents wind stimulation according to the magnitude of the applied voltage.

### Operation of Blink Acceleration Device According to One Embodiment

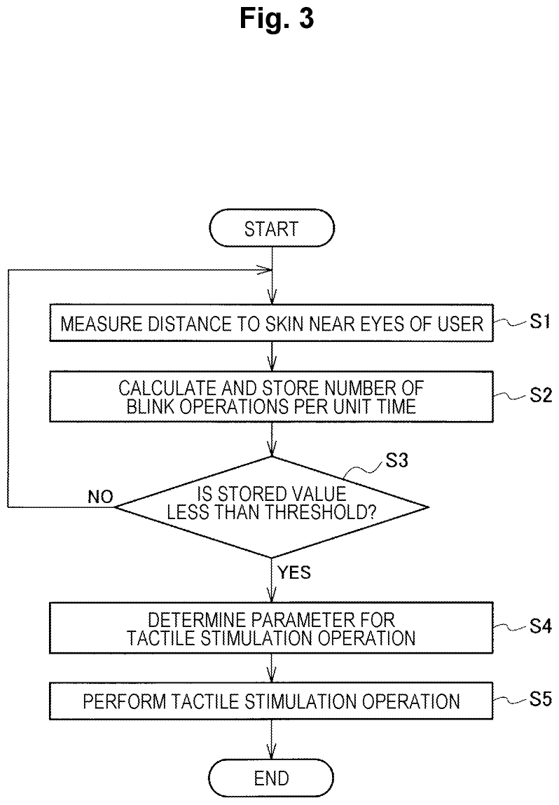

Next, an operation of the blink acceleration device 1 according to the present embodiment will be described with reference to the flowchart of FIG. 3.

First, with the user wearing the glasses 2, the photo reflector 111 measures the distance to the skin near the eyes of the user (S1). The photo reflector 111 outputs numerical values of 0 to 1023 according to the measured distance.

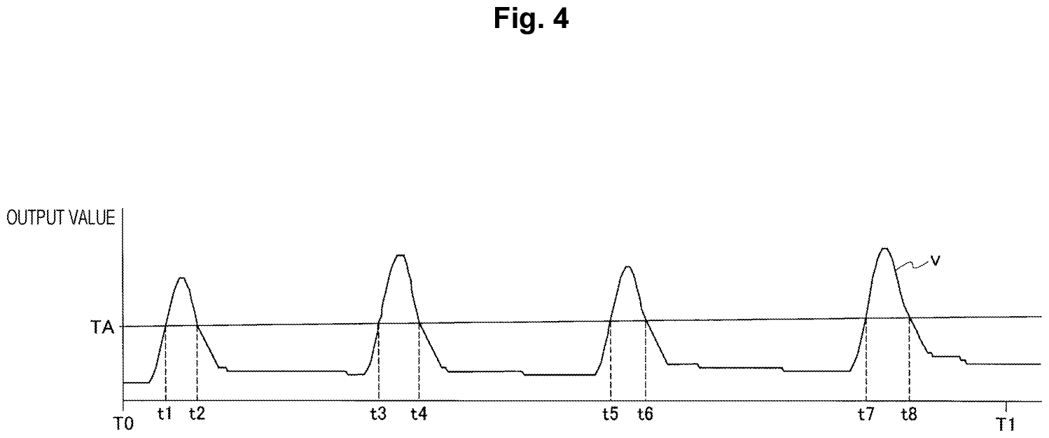

FIG. 4 illustrates an example of a temporal change in the output value by the photo reflector 111. Here, when the user blinks, the skin deforms, and an output value v changes across a preset threshold TA.

Next, the number of blinks calculation unit 141 of the microcomputer 14 calculates the number of blink operations per unit time on the basis of the value output from the photo reflector 111. Here, when the blink operation occurs once, the output value v and the threshold TA intersect each other twice when the user's skin is deformed by blinking and when the user's skin returns to the original state. Therefore, a value of ½ of the number the output value v of the photo reflector 111 intersects the threshold TA within a predetermined time is calculated as the number of blink operations per unit time.

In FIG. 4, the timing at which the output value v intersects the threshold TA occurs eight times from t1 to t8 between the times T0 to T1, and thereby the number of blink operations is calculated to be four. Information on the calculated number of blink operations is stored in the number of blinks storage unit 142 (S2).

Next, the operation control unit 143 determines whether the number stored in the number of blinks storage unit 142 is less than a preset threshold TB (S3). This threshold TB is set, for example, with the number of blinks required within a predetermined time to prevent dry eye symptoms.

In a case where the value stored in step S3 is less than the threshold TB (“YES” in S3), the operation control unit 143 determines that a tactile stimulus operation to the user is necessary, and determines a parameter related to the operation of the operation drive unit 13 (S4). This parameter is, for example, information related to a combination of the intensity, the duration, and the frequency regarding the blowing operation by the microblower 121 or a combination of the magnitude and the duration of the voltage applied to the motor driver 131, and is determined such that the tactile stimulus operation to the user becomes stronger as the number of blink operations is smaller.

Then, the motor driver 131 is driven by the parameter determined by the operation control unit 143, and the microblower 121 applies wind stimulation to the eyelashes of the user (S5). By operating the microblower 121 to stimulate the user's eyelashes and causing the eyelash reflex, blinking can be accelerated without drying the eyes.

When it is determined in step S3 that the number stored in the number of blinks storage unit 142 is less than the threshold TB (“NO” in S3), the operation control unit 143 determines that the tactile stimulus operation for the user is not necessary, and the process returns to step S1.

According to the above embodiment, it is possible to accelerate blinking and prevent dry eye without disturbing the user's work due to inhibition of vision and hearing. Furthermore, by using the reflection phenomenon that does not pass through the cerebral cortex, blinking can be accelerated without applying a cognitive load to the user as much as possible. Furthermore, reflection (eyelash reflex) to a tactile stimulus to the eyelashes can be induced with a lower stimulation intensity than that in a case of corneal reflex by a tactile stimulus to the vicinity of the outer corners of the eyes, so that blinking can be accelerated with suppressed voltage applied to a tactile actuator for presenting a tactile stimulus and low power consumption operation.

As a prototype, a glasses-type device in which a microblower is driven at a voltage of 5 V was manufactured by a microcomputer and a motor driver, and when wind stimulation was presented to one subject, corneal reflex did not occur and blinking did not occur due to weak stimulation in the wind stimulation to the vicinity of the outer corners of the eyes, but when wind stimulation to the roots of the eyelashes was presented, blinking occurred with a tickle feeling.

In the embodiment described above, a case where the photo reflector 111 is used as the operation state acquisition unit 11 has been described, but the present invention is not limited thereto. For example, information indicating a motion state of the skin near the eyes of the user may be acquired using an analysis device that analyzes imaging information obtained by imaging the vicinity of the eyes of the user with a camera device or a measuring instrument that measures an eye potential or a muscle potential of an electrode mounted near the eyes of the user.

In the embodiment described above, a case where a tactile stimulus is given to the user using the microblower 121 as the tactile stimulus operation unit 12 has been described, but the present invention is not limited thereto, and the tactile stimulus operation unit 12 may include at least one of a solenoid, a vibration motor, an electrical stimulation device, an ultrasonic speaker device, and a Peltier element.

In a case where a solenoid is used as the tactile stimulus operation unit 12, a contact stimulation is given to the user by a pin of the solenoid. In a case where a vibration motor is used as the tactile stimulus operation unit 12, a tactile stimulus by vibration is given to the user. In a case where an electrical stimulation device is used as the tactile stimulus operation unit 12, an electrode is attached near the eyes of the user, and a current flows through the electrode, thereby providing electrical stimulation to the user. In a case where an ultrasonic speaker device is used as the tactile stimulus operation unit 12, ultrasonic stimulation is applied to the vicinity of the eyes of the user. In a case where a Peltier element is used as the tactile stimulus operation unit 12, a temperature stimulus is given to the vicinity of the eyes of the user.

### Hardware Configuration

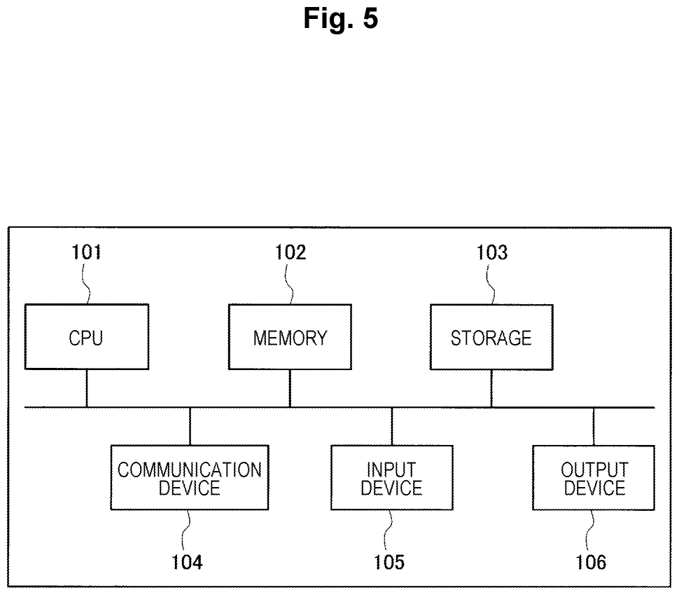

The microcomputer 14 of the blink acceleration device 1 described above can use, for example, a general-purpose computer system as illustrated in FIG. 5. The illustrated computer system includes a central processing unit (CPU, processor) 101, a memory 102, a storage 103 (hard disk drive (HDD), solid state drive (SSD)), a communication device 104, an input device 105, and an output device 106. The memory 102 and the storage 103 are storage devices. In this computer system, the CPU 101 executes a predetermined program loaded on the memory 102 to implement each function of the blink acceleration device 1.

The blink acceleration device 1 may be implemented by one computer, or may be implemented by a plurality of computers. The blink acceleration device 1 may be a virtual machine mounted on a computer.

The program for the blink acceleration device 1 can be stored in a computer-readable recording medium such as an HDD, an SSD, a universal serial bus (USB) memory, a compact disc (CD), or a digital versatile disc (DVD), or can be distributed via a network.

Although the embodiments of the present invention have been described above, the embodiments are presented as examples, and are not intended to limit the scope of the invention. This novel embodiment can be implemented in various other forms, and various omissions, substitutions, and changes can be made without departing from the gist of the invention. These embodiments and modifications thereof are included in the scope and gist of the invention, and are included in the invention described in the claims and the equivalent scope thereof.

### REFERENCE SIGNS LIST

1 Blink acceleration device
2 Glasses
11 Operation state acquisition unit
12 Tactile stimulus operation unit
13 Operation drive unit
14 Microcomputer
102 Memory
103 Storage
104 Communication device
105 Input device
106 Output device
111 Photo reflector
121 Microblower
131 Motor driver
141 Number of blinks calculation unit
142 Number of blinks storage unit
143 Operation control unit

1. A blink acceleration device comprising:
a number of blinks calculation unit, including one or more processors, configured to calculate a number of blink operations per unit time of a monitoring target person; and
a tactile stimulus operation unit, including one or more processors, configured to perform an operation of stimulating eyelashes as a tactile stimulus operation for the monitoring target person when the number of blink operations calculated by the number of blinks calculation unit is less than a preset threshold.

2. The blink acceleration device according to claim 1,
wherein the tactile stimulus operation unit is configured to perform the operation of stimulating eyelashes using wind stimulation.

3. The blink acceleration device according to claim 1,
wherein when the number calculated by the number of blinks calculation unit is less than the preset threshold, the tactile stimulus operation unit is configured to perform, based on the number, the operation of stimulating eyelashes with a parameter determined with respect to a combination of an intensity, a duration, and a frequency related to the operation of stimulating eyelashes of the monitoring target person or a combination of a magnitude of a voltage to be applied and the duration.

4. The blink acceleration device according to claim 3,
wherein the tactile stimulus operation unit is configured to determine the parameter such that tactile stimulus to the monitoring target person becomes stronger as the number calculated by the number of blinks calculation unit is smaller.

5. A blink acceleration method comprising:
calculating, by a blink acceleration device, a number of blink operations per unit time of a monitoring target person, and
when the number that has been calculated is less than a preset threshold, performing, by the blink acceleration device, an operation of stimulating eyelashes as a tactile stimulus operation for the monitoring target person.

6. The blink acceleration method according to claim 5, comprising:
performing the operation of stimulating eyelashes using wind stimulation.

7. The blink acceleration method according to claim 5, comprising:
when the number calculated by the number of blinks calculation unit is less than the preset threshold, performing, based on the number, the operation of stimulating eyelashes with a parameter determined with respect to a combination of an intensity, a duration, and a frequency related to the operation of stimulating eyelashes of the monitoring target person or a combination of a magnitude of a voltage to be applied and the duration.

8. The blink acceleration method according to claim 7, comprising:
determining the parameter such that tactile stimulus to the monitoring target person becomes stronger as the number calculated by the number of blinks calculation unit is smaller.
